(19) United States
(12) Plant Patent Application Publication
(10) Pub. No.: US 2016/0029531 P1
(43) Pub. Date: Jan. 28, 2016

(54) Ligustrum 'NCLX1'

(50) ;

(71) Applicant: North Carolina State University, Raleigh, NC (US)

(72) Inventor: Thomas G. Ranney, Raleigh, NC (US)

(21) Appl. No.: 14/121,065

(22) Filed: Jul. 25, 2014

(51) Int. Cl. A01H 5/00

(52) U.S. Cl. PLT/226

(57) ABSTRACT

‘NCLX1’ is a new cultivar of privet with bright, yellow-colored foliage, compact form with dense branching habit, fragrant white flowers from late-May to June, and low-fertility with limited fruit set.

### LATIN NAME OF THE GENUS AND SPECIES

The Latin name of the novel plant variety disclosed herein is Ligustrum.

### VARIETY DENOMINATION

The inventive variety of Ligustrum disclosed herein has been given the varietal denomination ‘NCLX1’.

### BACKGROUND OF THE INVENTION

The present invention comprises a new and distinct variety of Ligustrum, hereinafter referred to by the cultivar name ‘NCLX1’. This new privet was developed through a breeding program at North Carolina State University, Mills River, N.C. ‘NCLX1’ was selected from a population of seedlings generated by hybridizing Ligustrum×vicaryi (unpatented) with Ligustrum tschonoskii ‘Little Thomas’ (unpatented). The first asexual propagation of ‘NCLX1’ was carried out in July 2010 by rooting stem cuttings at the North Carolina State University, Mountain Horticultural Crops Research Station, Mills River, N.C. and ‘NCLX1’ has been asexually reproduced repeatedly by vegetative cuttings over a 3-year period. ‘NCLX1’ roots readily from softwood cuttings treated with a basal dip of 2,500-5,000 ppm indole butyric acid (potassium salt) in water. ‘NCLX1’ has been found to retain its distinctive characteristics through successive asexual propagations.

### SUMMARY OF THE INVENTION

The following are the most outstanding and distinguishing characteristics of this new cultivar when grown under standard horticultural practices at North Carolina State University, Mountain Horticultural Crops Research Station, Mills River, N.C.

1. Bright, yellow-colored foliage.

2. Compact form with dense branching habit.

3. Fragrant white flowers from late-May to June.

4. Low-fertility with limited fruit set.

### BRIEF DESCRIPTION OF THE DRAWINGS

This new privet is illustrated by the accompanying photographs which show the plant's form, foliage and inflorescences. The colors shown are as true as can be reasonably obtained by conventional photographic procedures. Colors in the photographs may differ slightly from the color values cited in the detailed botanical description, which accurately describe the colors of the new privet.

FIG. 1 shows the overall compact habit of a container-grown plant of ‘NCLX1’.

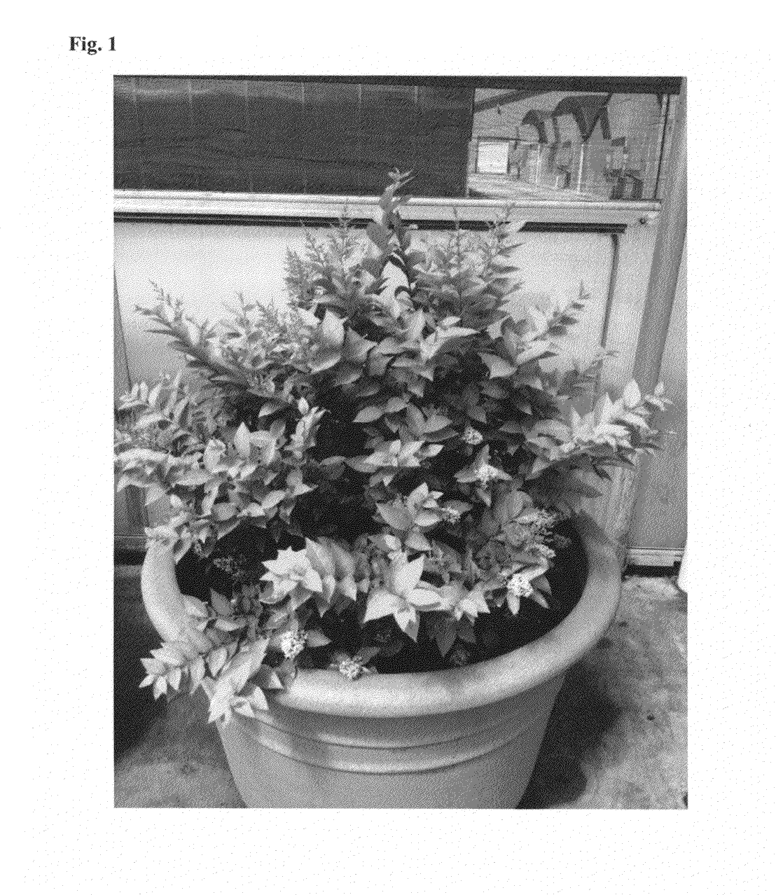

FIG. 2 shows the yellow foliage color of ‘NCLX1’.

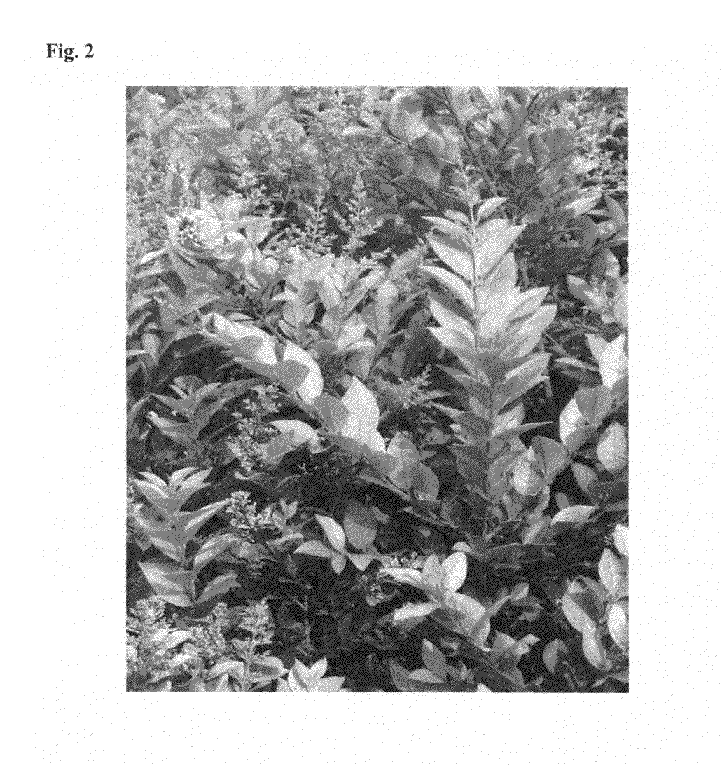

FIG. 3 shows the white flowers of ‘NCLX1’ and the compactness of the inflorescence.

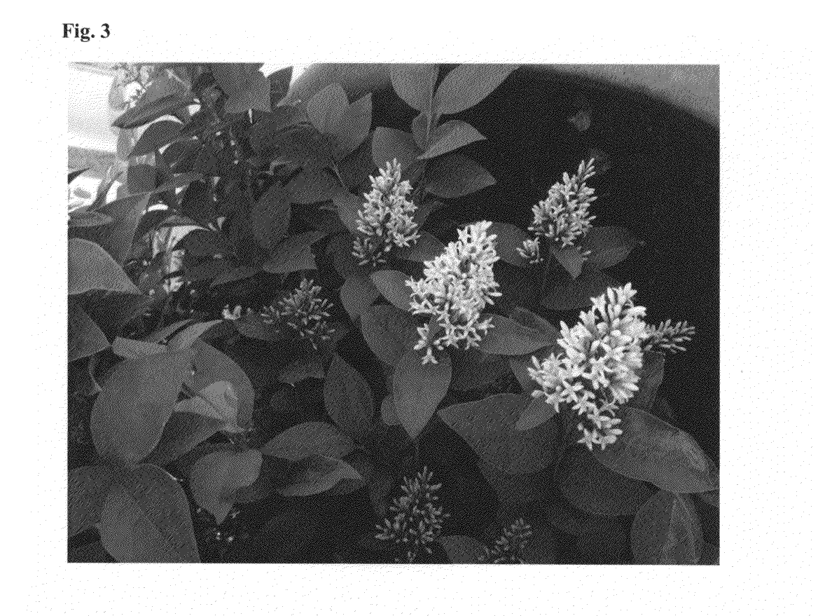

### DETAILED BOTANICAL DESCRIPTION

The following detailed description sets forth the distinctive characteristics of ‘NCLX1’. The detailed description was taken on 6-year-old plants growing in Mills River, N.C. in May 2014. All colors cited herein refer to The Royal Horticultural Society Colour Chart (The Royal Horticultural Society (R.H.S.), London, 2001 Edition). Where specific dimensions, sizes, colors, and other characteristics are given, it is to be understood that such characteristics are approximations or averages set forth as accurately as practicable.
Classification:
   Botanical name.—Ligustrum ‘NCLX1’.
   Common name.—Privet.
Parentage:
   Ligustrum×vicaryi×Ligustrum tschonoskii ‘Little Thomas’.
Plant description:
   Growth habit.—Upright to rounded with compact, dense branching habit.
   Height.—1.22-1.83 m at maturity.
   Width.—1.22-1.83 m at maturity.
Stem:
   Immature stem texture.—Glabrous.
   Immature stem color.—Yellow-green (144A) flushed with purple (N77C).
   Mature stem texture.—Glabrous, lenticelled.
   Mature stem color.—Greyed-green (197A).
   Lateral branch length.—Average 60 cm, range 30 to 100 cm.
   Internode length.—Average 2 cm, range 1 to 5 cm.
Leaves:
   Persistence.—Deciduous.
   Arrangement.—Opposite, simple.
   Length.—Average 3.2 cm, range 1.8 to 4.2 cm.
   Width.—Average 1.8 cm, range 1 to 2.4 cm.
   Shape.—Elliptical to ovate.
   Apex.—Acute.
   Base.—Rounded.
   Venation pattern.—Pinnate.
   Margin.—Entire.
   Emerging leaves.—Color: Upper and lower: Yellow-green (144A to N144A). Texture: Glabrous.
   Mature leaves during growing season.—Color: Upper: Yellow-green (N144A). Lower: Yellow-green (145A). Venation: Upper: Yellow-green (N144A). Lower: Yellow-green (N145). Texture: Glabrous.
   Petiole.—Length: Average 2.5 mm, range 1.0 to 3.5 mm. Width: Average 0.7 mm, range 0.6 to 0.9 mm. Texture: Glabrous. Color: Yellow-green (N144A).
Inflorescence:
   Description.—Terminal panicle of racemes with multiple flowers.
   Number of flowers per inflorescence.—30-50.
   Flowering season.—late May to June.
   Flower longevity.—10-14 days.
   Fragrance.—Scented.
   Inflorescence height.—Average 4 cm, range 2-6 cm.
   Inflorescence diameter.—Average 2.5 cm, range 2-3 cm.
   Flower diameter.—Average 5.0 mm, range 4.0-6.0 mm.
   Flower depth.—Average 6.0 mm, range 5.0-7.0 mm.
   Flower buds.—Length: Less than 8.0 mm. Diameter: Less than 3.0 mm. Shape: Oblong. Color: White (155B).
   Petals.—Quantity: Four per flower. Length: About 3.0 mm. Width: About 2.0 mm. Shape: Oblong, recurved. Apex: Acute. Base: Fused. Texture: Glabrous. Color: White (155B).
   Sepals.—Quantity: Four per flower. Length: About 3.0 mm. Width: About 1.5 mm. Apex: Acute. Base: Fused. Texture: Glabrous. Color: Yellow-green (145A).
   Peduncle.—Length: About 5.0 mm. Width: About 2.0 mm. Texture: Glabrous. Color: Green (143C).
   Pedicels.—Length: About 1.5 mm. Width: About 1.0 mm. Texture: Glabrous. Color: Green (143C).
Fruit:
   Description.—Berry-like drupe.
   Frequency.—Infrequent, very limited fruit set compared to parental taxa.
   Length.—About 7.0 mm. Width: About 5.0 mm. Shape: Oblong. Texture: Glabrous.
   Color.—Black (202A).
Disease and insect resistance: No significant disease or insect pests have been observed.
Cold hardiness: At least USDA zone 6b; testing has not been completed in colder zones.

### COMPARISON WITH PARENTAL SPECIES

Table 1 shows key distinguishing characteristics between ‘NCLX1’ and its parental taxa. ‘NCLX1’ is distinguished from Ligustrum×vicaryi in that ‘NCLX1’ has more compact inflorescences, a more compact habit, and has very limited fruit production. ‘NCLX1’ is distinguished from Ligustrum tschonoskii ‘Little Thomas’ by having bright yellow mature leaf color and later flowering time.

TABLE 1

| | Taxa | | |
|---|---|---|---|
| | | Ligustrum | |
| | Ligustrum | tschnoskii | |
| Trait | xvicaryi | ‘Little Thomas’ | Ligustrum ‘NCLX1’ |
| Growth habit | Vase-shaped, | Rounded, spreading | Upright, |
| | somewhat rangy | shrub | compact shrub |
| | shrub | | |
| Mature size | 10-12′ tall × 8-10′ | 4-6′ tall × 6-8′ wide | 4-6′ tall × 4-6′ wide |
| | wide | | |
| Emerging foliage | Upper: Yellow- | Upper and Lower: | Upper and Lower: |
| color | green (N144A) | Yellow-green | Yellow-green (144A to |
| | Lower: Yellow- | (144A) | N144A) |
| | green (146D) | | |
| Mature foliage color | Upper and lower: | Upper: Green | Upper: Yellow-green |
| | Yellow-green | (138A) | (N144A) |
| | (151A) | Lower: Yellow- | Lower: Yellow-green |
| | | green (146B) | (145A) |
| Flowering season | Early to mid-June | Mid- to late May | Early to mid-June |
| Number of flowers | About 100, ranges | About 45, ranges | About 35, ranges from |
| per inflorescense | from 50 to 180 | from 25 to 90 | 10 to 80 |
| Inflorescence height | About 6 cm, ranges | About 4 cm, ranges | About 4 cm, ranges |
| | from 4 to 7 cm | from 2 to 6 cm | from 2 to 6 cm |
| Fragrance | Light, sweet | Light, acrid | Light, sweet |
| | fragrance | fragrance | fragrance |
| Seed production | Prolific | Prolific | Infrequent |

What is claimed is:

1. A new and distinct privet plant named ‘NCLX1’ substantially as illustrated and described herein.
